(19) United States
(12) Patent Application Publication
(10) Pub. No.: US 2026/0000303 A1
(43) Pub. Date: Jan. 1, 2026

(54) Intuitively and rapidly applicable tourniquets

(71) Applicants: Michael J. Dimino, FREEHOLD, NJ (US); Michael C. Dimino, FREEHOLD, NJ (US); Alfonse Dimino, FREEHOLD, NJ (US)

(72) Inventors: Michael J. Dimino, FREEHOLD, NJ (US); Michael C. Dimino, FREEHOLD, NJ (US); Alfonse Dimino, FREEHOLD, NJ (US)

(21) Appl. No.: 18/945,506

(22) Filed: Mar. 24, 2025

Related U.S. Application Data
(63) Continuation-in-part of application No. 17/180,931, filed on Feb. 22, 2021, now Pat. No. 12,185,952.
Application No. 17/180,931 is a continuation-in-part of application No. 15/833,626, filed on Dec. 6, 2017, now Pat. No. 10,925,617.
Application No. 15/833,626 is a continuation-in-part of application No. 29/579,266, filed on Sep. 29, 2016, now Pat. No. D825752.
Application No. 29/579,266 is a continuation-in-part of application No. 29/607,446, filed on Jun. 13, 2017, now Pat. No. D891614.
Application No. 17/180,931 is a continuation-in-part of application No. 15/932,437, filed on May 22, 2017, now abandoned.
(60) Provisional application No. 62/496,016, filed on Oct. 3, 2016.
(60) Provisional application No. 62/496,017, filed on Oct. 3, 2016.
(60) Provisional application No. 62/496,018, filed on Oct. 3, 2016.

(51) Int. Cl. A61B 5/0205; A61B 5/00; A61B 5/01; A61B 5/02; A61B 5/024; A61B 5/11; A61B 5/1455; A61B 17/00; A61B 17/132; G16H 40/67; G16H 50/20

(52) U.S. Cl. CPC A61B 5/02055; A61B 5/0022; A61B 5/01; A61B 5/02042; A61B 5/02422; A61B 5/1112; A61B 5/14552; A61B 5/7264; A61B 5/7275; A61B 5/746; A61B 17/1325; G16H 40/67; G16H 50/20; A61B 2017/00057; A61B 2560/0214

(57) ABSTRACT

The Retrofit Tourniquet System is a portable, non-invasive diagnostic device designed to assess and monitor vital signs for early detection of hypovolemic shock and other trauma-related medical conditions. Utilizing integrated sensors, the system continuously tracks critical metrics such as heart rate, blood pressure, respiratory rate, pulse pressure, and blood oxygen levels, comparing them against individualized baseline values stored in lookup tables. The system's diagnostic algorithms identify deviations in vital signs, calculate estimated blood loss stages, and provide recommended treatment options. Built to function seamlessly on or off a tourniquet, the Retrofit Tourniquet System enables first responders and military personnel to assess a patient's condition in real-time, even in field conditions. Enhanced by secure data encryption and compatibility with healthcare interoperability standards (e.g., FHIR and HL7), the system also supports remote monitoring by medical professionals when integrated with remote databases. This tailored, AI-assisted device improves patient survivability by allowing for timely interventions, particularly in cases of internal bleeding where traditional diagnostics are insufficient.

### CROSS-REFERENCE TO RELATED APPLICATION

The present patent application is a continuation-in-part of patent application Ser. No. 17/180,931, filed Feb. 22, 2021, which is a continuation-in-part under 37 C.F.R. § 1.53(b) of prior U.S. patent application Ser. No. 15/833,626, filed Dec. 6, 2017, now U.S. Pat. No. 10,925,617, issued Feb. 23, 2021, by Michael J. DIMINO, Michael C. DIMINO and Alfonse DIMINO, and entitled “INTUITIVELY AND RAPIDLY APPLICABLE TOURNIQUETS,” which claims the benefit of and priority to U.S. Provisional Patent Application No. 62/509,614, filed May 22, 2017. U.S. patent application Ser. No. 15/833,626 is also a continuation-in-part of U.S. Design patents application Ser. Nos. 29/579,266, filed Sep. 29, 2016, now U.S. Design Pat. No. D825,752S, issued Aug. 14, 2018 and Ser. No. 29/607,446, filed Jun. 13, 2017, now U.S. Design Pat. No. D891,614S, issued Jul. 28, 2020. The present patent application is also a continuation-in-part of U.S. patent application Ser. No. 15/932,437, filed May 22, 2017, by Michael J. DIMINO, Michael C. DIMINO and Alfonse DIMINO, and entitled “TOURNIQUET,” which claims the benefit of and priority to U.S. Provisional Patent Application Nos. 62/496,016, filed Oct. 3, 2016, 62/496,017, filed Oct. 3, 2016 and 62/496,018, filed Oct. 3, 2016. The entire contents of each of the patent applications listed above are incorporated herein by reference.

### BACKGROUND OF THE INVENTION

The Retrofit Tourniquet System was developed in response to the urgent need for a pre-hospital diagnostic device capable of assessing and monitoring vital health indicators in trauma patients, particularly in high-stress or remote environments such as military combat zones or field emergency response areas. Hypovolemic shock, often due to severe blood loss, is a leading cause of preventable death in trauma settings. For soldiers and other individuals in similar situations, hypovolemic shock can be challenging to diagnose quickly, and current portable diagnostic options are limited. Alarmingly, over 62% of bleeding soldiers perish from hypovolemic shock before they reach a hospital, highlighting the need for immediate on-site intervention.

The Retrofit Tourniquet System is designed to detect early signs of hypovolemic shock and other related conditions by continuously monitoring vital signs. Unlike traditional diagnostic tools that may not capture internal bleeding without visible symptoms, this system provides a non-invasive, portable, and accurate means to detect blood loss and related health risks. The system is versatile, functioning effectively both as an integrated part of a tourniquet and independently, allowing users to gauge critical health parameters and predict shock risk before evacuation to medical facilities.

Additionally, the Retrofit Tourniquet System uses diagnostic algorithms and age- and condition-specific lookup tables to assess variations in vital signs accurately, adapting its analysis to the specific health profiles of individual users. This enables the system to recognize subtle shifts in vital signs and trigger alerts or recommend interventions tailored to each patient's unique needs.

The device is also built with robust security and data interoperability, ensuring compliance with privacy standards such as HIPAA. It uses encrypted connections and aligns with healthcare interoperability standards like FHIR and HL7 to securely connect with remote medical databases, making it a safe, adaptable tool for military and civilian medical use alike.

The Retrofit Tourniquet System thus provides a comprehensive, innovative solution for rapid, on-site diagnostics and intervention in trauma cases, with the potential to save lives and enhance patient outcomes in emergency medical settings.

### SUMMARY OF INVENTION

It is the object of the invention to provide a means of diagnosing hypovolemic shock and other medical conditions associated with hypovolemic shock. It is a further object of the invention to provide a diagnostic device that can be used on or off a tourniquet. It is yet a further object of the invention to determine how much blood has been lost based on a lookup table of different stages of blood loss based on current vital signs and provide a recommended treatment.

There is a need for a portable non-invasive method to determine the medical condition of a patient before they are sent to a hospital; in the field more than 62% of bleeding soldiers will die from hypovolemic shock before they reach a hospital. There needs to be a device that soldiers and medics can use in the field to know the probability of hypovolemic shock stages so the proper intervention can begin before the patient is EVAC to a medical facility. The sensor system proves particularly valuable for diagnosing internal bleeding, as it detects reading levels indicative of internal bleeding even when no external bleeding is visible.

The System, Continuous Monitoring of Vital Signs—The system regularly scans current vital signs (e.g. heart rate, blood pressure, respiration, pulse pressure, blood oxygen levels) using integrated sensors. These measurements are compared against a set of pre-programmed thresholds stored in a lookup table.

Deviation Detection Using Lookup Tables—The lookup tables provide baseline ranges for various vital signs, taking into account typical variations by age, gender, and specific medical conditions. When a deviation from these baselines is detected, the system identifies it as an anomaly or potential health concerns.

Threshold-Based Alarms—The system triggers an alarm or flag when measurements fall outside acceptable ranges, allowing for rapid intervention if there's a potentially serious condition.

### Evaluation of Blood Loss Levels

Blood loss Detection Algorithm—Upon detecting a significant deviation in vital signs that could indicate blood loss (such as a sudden drop in blood pressure, increased heart rate, or low oxygen saturation), the system activates a blood loss evaluation process.

### Comparison Against Historical Data—The System Uses the Individual

Historical vital signs data stored in the lookup table to differentiate between normal variability and symptoms potentially indicative of blood loss. For example, it might compare recent readings with the person's typical blood pressure or pulse levels to assess if the changes are consistent with blood loss.

Continuous Monitoring for Stability—The system continues monitoring to verify whether these deviations stabilize or worsen, helping to confirm the accuracy of a suspected blood loss condition.

The system utilizes multiple lookup tables that include baseline data adjusted for variables like skin thickness (which can affect the accuracy of certain sensor readings, such as pulse oximetry), age, gender, and pre-existing medical conditions (e.g., diabetes, hypertension).

Age and Condition Specific Baselines—By referencing these specialized tables, the system can account for age-related changes in vital signs or the impact of conditions that affect blood circulation, like thin skin or chronic illnesses.

The system cross-references real-time data with these lookup tables, adjusting its diagnostic criteria based on the specific profile of the individual. For example, a young, healthy individual's blood pressure range may differ from that of an older adult with hypertension, and the system takes this into account when evaluating deviations.

Diagnostic Algorithms—The system integrates diagnostic algorithms to correlate the observed deviations with known symptoms of relevant medical conditions. For example, a combination of low blood pressure and high heart rate might be associated with shock or severe blood loss, prompting the system to investigate further.

Medical Condition Matching—By matching symptoms with common medical profiles in its database, the system narrows down potential causes. This allows it to account for conditions that may not present as immediately obvious but require specific attention based on vital sign patterns.

Automated Treatment Suggestions—Once the system has processed and correlated the data, it generates a treatment recommendation. This might include suggesting emergency interventions (such as IV fluids in cases of suspected blood loss or shock), medication options, or next steps for medical personnel to follow. Also, amount of fluids that is needed for IV transfusion based on stage of blood loss determined by the algorithms.

Personalized Treatment Plans—The treatment recommendation is tailored to the individual's specific conditions. For example, for a patient with a history of heart disease, the system may adjust treatment suggestions based on risks associated with certain interventions.

Real-Time Update and Recommendations—If the individual's condition changes, the system can continuously re-evaluate the data and update its recommendations accordingly.

Sensor Data—Signals using various algorithms and lookup tables, hypovolemic shock, blood loss, and other medical conditions can be diagnosed. These vital signs help in developing diagnostic markets, enabling the detection and assessment of a range of medical conditions and blood loss scenarios.

Data Storage for Historical Vital Signs—Purpose, the primary goal of storing historical data is to create a personal baseline for each individual. This enables the system to recognize normal versus abnormal patterns for each specific person, rather than relying on generalized averages.

Architecture—The storage solution should be secure and scalable, likely based on a cloud or hybrid-cloud setup, allowing access from multiple locations if needed.

Data Granularity—This data would include individual metrics like heart rate, blood pressure, respiratory rate, and oxygen saturation, recorded at intervals over time. Ideally, the data would also capture conditions under which these readings were taken, such as resting or active states.

Edge Processing for Speed—To reduce latency and data volume, some initial processing might occur directly on the device (edge processing). This means the device could filter out less relevant data or flag potential issues even before sending the information to the main system

Machine Learning Algorithms—Using machine learning, the system could detect complex patterns in health metrics over time. For example, subtle shifts in data that may indicate early warning signs of illness or stress, even if the data doesn't immediately cross critical thresholds, could be identified.

Deviation Detection and Alerting—Deviation Criteria; Deviation is not just a simple threshold breach. Algorithms can use time-series analysis to detect gradual changes that may still indicate health issues. For instance, a steady rise in heart rate over several days might signal stress, dehydration, or another concern, even if it's not an acute event.

If a deviation is detected, the system would follow a pre-defined alerting protocol. Alerts might vary depending on the severity of the deviation minor fluctuations could be logged and monitored, while major deviations could trigger immediate alerts to medical personnel or even automated responses.

Such a system could significantly improve early detection of health issues, allowing for timely interventions that enhance passenger safety and care. This kind of proactive approach could be especially useful in high stress environments or for individuals with specific health concerns.

Remote Monitoring of Patients—Geofencing and Location Monitoring, GPS Based Geofencing. The system would create a virtual perimeter around the quarantine area (e.g. the individual's home or designated quarantine facility) using GPS technology. This geofence sets the boundaries that the quarantined individual should not cross.

Automated Location Tracking—The system would periodically update the individual's location in real time. If the person remains within the designated geofence, data is simply logged, ensuring privacy and maintaining compliance.

Boundary Alerts—If the individual crosses the geofenced boundary, the system immediately triggers an alert. This could include sending an alarm with the individual's location to a monitoring station, healthcare provider, or designated quarantine authorities.

Continuous Vital Signs Tracking—The system would continuously monitor the individual's vital signs using medical sensors. These could include heart rate, temperature, respiratory rate, oxygen saturation levels, which are critical for identifying symptoms or deterioration.

For some contagious conditions, specific vitals or symptoms (such as blood oxygen levels for respiratory illnesses) might need closer tracking. The system can be customized to focus on these metrics, adjusting alert thresholds based on the known progression of the condition.

Regular Data Transmission—The system would be set to send periodic updates to a central monitoring station automatically, including the individual's location, vital signs, and any notable health changes. This allows healthcare providers or quarantine officials to stay informed about the individual's status without requiring them to be physically present.

If the individual's condition worsens or if they leave the quarantine area, the system could adjust the frequency of data collection and transmission to capture more detailed information in near real-time, ensuring a rapid response if needed.

Geofence Breach Alert—Upon detecting that the person has crossed the geofence, the system would send an alert to the central monitoring station. This alert could include:

Location and Time of Breach—Where and when the breach occurred.

Real-Time Tracking—The individual's ongoing location to help track movement and potential risks to others.

Vital Signs Status—The individual's current health condition, as indicated by the most recent sensor data.

Health Alert Protocols—If the system detects abnormal vital signs such as a fever spike or low oxygen saturation, automated alerts can notify the monitoring station of potential health concerns, triggering intervention protocols.

Data from multiple quarantined individuals can be monitored through a centralized dashboard. This dashboard could offer filtering options to focus on those showing signs of deterioration, recent geofence breaches, or other priority conditions.

Incident Logging—The system logs all activities, such as geofence breaches, health alerts, and periodic check-ins. This creates a documented record, which could be useful for contact tracing, legal compliance, or post incident analysis.

For geofence breaches or health crises, the system would automatically notify local response teams, allowing them to reach the individual quickly.

Secure Data Transmission—All data, including location and health data, would be encrypted to protect patient privacy, complying with relevant privacy laws and regulations.

Controlled Data Access—Only authorized personnel, such as healthcare providers and designated monitoring authorities, would have access to the data, with strict access controls in place.

The system could be configured to retain data for the duration of the quarantine and then either archive or delete it, as required by regulatory guidelines.

Benefits and Applications of the Quarantine Monitoring System—Increased Safety and Compliance, by monitoring both location and health metrics, the system helps ensure that individuals comply with quarantine requirements, reducing the risk of spreading the condition.

Reduced Need for In-Person Checks—Continuous remote monitoring minimizes the need for physical check-ins, which helps protect healthcare workers and reduces the strain on medical resources.

Faster Response to Health Deterioration—The system's health monitoring allows authorities to detect and respond to worsening conditions early, ensuring timely care and preventing complications.

This system would be especially valuable in managing public health during outbreaks of highly contagious diseases, allowing authorities to maintain effective quarantine measures while protecting patient privacy and ensuring patient safety.

The system would connect to remote AI medical databases through secure network protocols and interoperability standards commonly used in healthcare, such as:

The system can use APIs (Application Programming Interfaces) to securely retrieve and send data to remote databases. APIs allows the system to access patient data, diagnostic resources, and other information from remote medical and specialized data banks.

FHIR and HL7 Standards: For compatibility with electronic health record (HER) systems, the system could use FHIR (Fast Healthcare Interoperability Resources) and HL7 standards. These are widely adopted standards in healthcare, enabling it to connect seamlessly with various medical databases and systems.

VPNs and Encrypted Connections: To ensure secure data transmission, the system could connect over Virtual Private Networks (VPNs) or other encrypted communication channels, protecting patient data and ensuring HIPAA compliance when transmitting sensitive information.

### BRIEF DESCRIPTION OF DRAWINGS

FIG. 1, Diagrammatically depicts the interior of a biosensor, sleeve housing with a finger inside of it being used.

FIG. 2, Diagrammatically depicts a sleeve housing with a tourniquet inserted through it.

FIG. 3, Depicts the outside of a biosensor sleeve with an O LED display.

### DETAIL DESCRIPTION OF DRAWING

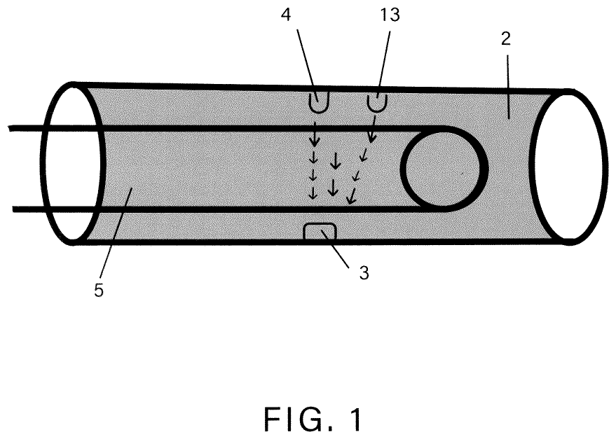

FIG. 1 diagrammatically depicts a finger 5 placed inside of system housing 2.2 LEDs 4, 13 shine light on finger directed to photodiode 3, both ends of housing 2 are open 6 & &.

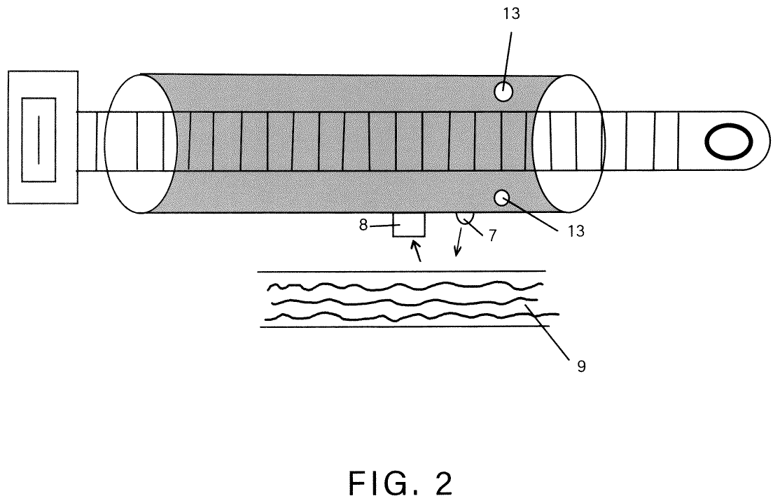

FIG. 2 depicts system housing and display 11. Attach to housing are two contact points 13 that are used for ECG sensors where 2 fingers are placed to acquire a ECG reading. A photodiode 8 receives reflected light, 7 green LED shines light onto body tissue 9. Both LEDs are attached to a tourniquet 10, is inserted into the exterior of housing sleeve 2.

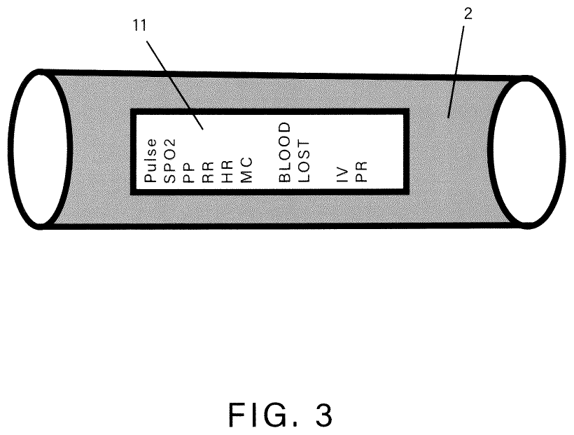

FIG. 3 shows housing enclosure 11 on system sleeve 2.

What is claimed is:

1. A retrofit tourniquet system comprising:
A system for diagnosing the probability or onset of hypovolemic shock and medical conditions associated with hypovolemic shock, and a variety of other medical conditions and blood loss amount based on vital signs and other physiological and biological parameters.
The retrofit tourniquet system comprises:
A plurality of sensors including heart rate sensors, blood pressure sensors, sweat biochemistry sensors, pulse pressure, temperature sensor, electrocardiogram sensor (ECG), blood oxygenation sensors, respiration rate, perfusion index, and pressure sensor.
The system may use vital sign values based on photoplethysmography (PPG) optical method, PPG waveform, pulse travel time (PTT), spectrophotometry, photoelectric oxyhemoglobin sensors physiological and biological sensors.
A portable wireless energy source coupled with the light source and for supplying energy to the system.
A look-up table with a set of predetermined anomalous and cardiac arrhythmia, vital sign threshold for each individual sensor that would indicate a specific medical condition.
A look-up table programmed with predetermined stages of blood loss amounts based on sensor data and vital sign values.
A look-up table of stored historical vital sign data.
A look-up table with predetermined anomalous and arrhythmia vital sign thresholds, based on age and gender.
A lookup table of amounts of transfusion liquids and type of transfusion-based on blood loss amounts.
A look-up table with a variety of medical conditions with predetermined vital signs that indicate such medical conditions.
A look-up table of recommended treatment for a specific medical condition.
A system consisting of light emitting diodes (LED) and photo diodes.
A GPS receiver for receiving position coordinates.
A processor and controller is configured to receive sensor data and compare such data to the corresponding look-up tables including medical condition, vital signs, blood loss amounts, amount of transfusion., and recommended treatment using algorithms and lookup tables
A warning system when predetermined thresholds are met.
A means to choose either reflective or transmissive mode of operation.
A cellular satellite and UHF-VHF transceiver for receiving and transmitting voice and data.

2. The retrofit tourniquet system of claim 1, sensors continuously monitor current vital signs if a deviation is detected from a predetermined set of values in a vital sign lookup table; the system initiates a scan of lookup tables indicating blood loss levels. Comparing these values with the current vital sign readings. The system then cross references another lookup table containing medical conditions associated with such deviations. The system further analyzes these readings relative to age and gender to assess and identify potential medical conditions based on cross-referenced data. The system generates a recommended treatment plan tailored to the identified conditions.

3. The retrofit tourniquet system of claim 1, By processing PPG signals with different algorithms and lookup tables, hypovolemic shock and other medical conditions can be diagnosed. The vital signs are used to develop diagnostic markers for a variety of medical conditions and blood loss amounts.

4. The retrofit tourniquet system of claim 1 contains Bluetooth and Wi-Fi.

5. The retrofit tourniquet system of claim 1 contains storage for all sensor data to compare past sensor data with current sensor data to monitor any deviation from past vital sign readings.

6. The retrofit tourniquet system of claim 1, the end user can program into the system their preexisting medical conditions so that the system can compensate for such condition.

7. The retrofit tourniquet system of claim 1, two methods can be used to acquire sensor data including reflective mode consisting of a green light emitting diode (LED) and photodetector, both positioned next to each other. Transmissive mode two red light emitting diodes (LED) and a photo detector positioned on opposite side of each other.

8. The retrofit tourniquet system of claim 1, consists of a thermometers and a combination of a thermopile and thermistor. The thermopile that produces an infrared radiation (Heat) focused onto one side of the thermopile, the amount of current it produces is determined by the difference in temperature. This reveals a relative difference in temperature a thermistor is used to determine the ambient skin temperature of the unheated metal in the thermopile. The thermistors are an electrical resistor whose resistance changes based on the temperature.

9. The retrofit tourniquet system of claim 1, the housing for all the sensors and GPS receiver and transceivers are housed on the top outer position of a flexible sleeve. The interior of the sleeve consists of a top portion ceiling and bottom portion floor. The top portion contains a red LED, and the bottom portion has a photodiode. This configuration is meant to read vitals from a finger that is placed into the interior of the sleeves.

10. The retrofit tourniquet system of claim 10, the system electronics is positioned exterior on top of upper section of a flexible sleeve. The sleeve interior comprises an upper and lower portion. The upper portion contains two LED, and the lower portion includes a photodiode. These components are configured to read vital signs from a user's finger. There are also two metal contacts that are used for ECG readings.

11. The retrofit tourniquet system of claim 1, contains a A green light emitting diode (LED) and photodiode are placed side by side on the bottom exterior of the sleeve facing a part of the human body part where the tourniquet is placed to occlude blood flow, this method is called reflective mode.

12. The retrofit tourniquet system of claim 1 is configured to scan current vital signs data and detect deviations based on preprogrammed thresholds from look-up table. Upon detecting a deviation, the system further evaluates blood loss levels by comparing current vital sign reading to blood loss amounts from a look-up table. The system accounts for conditions involving thin skin and associated medical condition by referencing multiple look-up tables. It then cross references this data with age, gender and preexisting medical conditions of the individual, processes the information, and correlates it to relevant medical conditions. The system then provides a treatment recommendation.

13. The retrofit tourniquet system of claim 1 can receives programming instructions in order to upgrade the system with new diverse trauma care discoveries and algorithms.

14. The retrofit tourniquet system of claim 1, is capable of predictive analysis for shock prevention. The system combined with a tourniquet or by itself can predict life threatening conditions based on initial data collected within minutes. The system can forecast the likely progression of blood loss and shock risk suggesting when intervention such as blood transfusion might be required.

15. The retrofit tourniquet system calculates oxygen saturation based on the difference in the absorption spectrum of hemoglobin. Two LEDs are used, red and infrared deoxyhemoglobin absorbs more light at 660 nano meters (NM) and at 940 NM oxygenated hemoglobin absorbs more light.

16. The retrofit tourniquet system of claim 1, consists of Electrocardiogram (ECG) sensors to record the electrical signals that control the heart rhythm. Looks for anomalous in vital sign from what is normal and past history.

17. The retrofit tourniquet system of claim 1, the system can connect to remote AI (artificial intelligence) medical data banks to transfer sensor data to the bank to enhance diagnostics through real-time data integration and advanced analytics.

18. The retrofit tourniquet system of claim 1, consists of A retro fitted tracking biosensor system for emergency response tourniquet, incorporating a decentralized mesh network and artificial intelligence (AI) machine learning (ML), wherein each device functions as a relay node to maintain continuous connectivity in off-grid areas. The device system includes a suite of biosensors capable of detecting and transmitting patient data—such as heart rate, blood pressure, blood oxygen levels, respiratory rate, body temperature, ECG, glucose levels, hydration status, and blood coagulation metrics, etc. . . . The AI/ML module continuously analyzes real-time and historical biosensor data to detect critical health events, including hypovolemic shock, hypoxia, and arrhythmias, and autonomously adjusts network communication based on data insights to facilitate efficient, off-grid emergency response as well as on the grid connectivity when available if need be.

19. The retrofit tourniquet system of claim 1, consists of a medical device system for emergency trauma care comprising a wearable tourniquet device module with GPS tracking and biosensor capabilities, an AI-powered network configured to transmit real-time patient health and location data, and an augmented reality (AR) interface on a VR style headset similar to Microsoft HoloLens or any desired display type. The device system overlays location, health status, and prioritized care instructions within the display, enabling first responders and medical personnel to visually locate, assess, and track patients in both military and civilian settings with predictive triage analysis. The network synchronizes data across devices, allowing the HoloLens to display the GPS location and triage status of multiple patients simultaneously, facilitating coordinated trauma care and rapid decision-making by emergency teams.

20. The retrofit tourniquet system of claim 1, consists of a wearable medical device system comprising a pulse biosensor configured to continuously monitor heart rate and pulse waveform characteristics, with an AI module that analyzes pulse data to detect indicators of hypovolemic shock in real time. The device utilizes a machine learning (ML) algorithm trained on historical data to recognize early-stage shock through deviations in pulse amplitude, waveform variability, and heart rate trends, transmitting alerts to responders via a mesh network upon detection of potential shock. AI-driven signal processing unit that enhances pulse data accuracy by filtering noise and calculating a shock index to quantify shock severity. The ML model adapts sensitivity thresholds based on individual patient baselines and environmental conditions, improving detection accuracy. Additionally, the AI prioritizes hypovolemic shock alerts by severity and routes real-time triage data through the mesh network to connected responders or augmented reality devices, facilitating rapid response and situational awareness.

21. The retrofit tourniquet system of claim 1, consists of a wearable medical device system comprising a microneedle array for transdermal drug delivery, an AI module that monitors biosensor data to determine optimal timing and dosage for drug release, and a nanoparticle-based drug formulation for controlled release. The AI/ML algorithm, enhanced by machine learning, adjusts drug dosages based on patient-specific metrics, such as pulse rate, blood pressure, and indicators of hypovolemic shock, ensuring precise, real-time therapeutic response tailored to the patient's condition. The device communicates administered doses and patient responses through a network or manual override, allowing coordinated monitoring and intervention by connected responders in emergency scenarios.

22. The retrofit tourniquet system of claim 1 consisting of a wearable tourniquet medical device system comprising a GPS module for real-time location tracking, a network communication module, and an autonomous drone with an AI/ML module that interprets biosensor data to assess patient needs. The drone is configured to locate the wearable device via GPS coordinates, deliver emergency supplies based on the AI-determined urgency, and provide situational awareness through heat and sound signature detection, aiding in trauma response. The drone's navigation is dynamically adjusted according to patient vitals and environmental factors, with a two-way communication system that relays real-time patient status to optimize supply delivery. Additionally, the AI module coordinates with other connected devices to prioritize and synchronize responses across multiple individuals in complex emergency scenarios.
